(19) United States
(12) Patent Application Publication
(10) Pub. No.: US 2010/0094078 A1
(43) Pub. Date: Apr. 15, 2010

(54) DEVICE AND METHOD FOR ENHANCING MILK LET-DOWN WITH A BREAST PUMP

(75) Inventor: Richard S. Weston, Encinitas, CA (US)

Correspondence Address: PROCOPIO, CORY, HARGREAVES & SAVITCH LLP, 530 B STREET, SUITE 2100, SAN DIEGO, CA, 92101, US

(73) Assignee: HYGEIA II MEDICAL GROUP, INC., Carlsbad, CA (US)

(21) Appl. No.: 12/578,967

(22) Filed: Oct. 14, 2009

Related U.S. Application Data
(60) Provisional application No. 61/196,179, filed on Oct. 14, 2008.

(51) Int. Cl. A61M 21/00; A61M 1/06

(52) U.S. Cl. 600/27; 604/74; 700/94

(57) ABSTRACT

A breast pump assembly includes a vacuum pump for creating suction within one or two breast shields to the vacuum pump, and electronic recording device comprising a microphone, a memory for storing the sound received via the microphone, and a playback activator. The sound module or sound chip can be used to record and playback sounds made by a woman's baby to enhance the let-down reflex during pumping to facilitate collection of milk.

### RELATED APPLICATIONS

This application claims the priority of U.S. Provisional Application No. 61/196,179, filed Oct. 14, 2008, which is incorporated herein by reference.

### FIELD OF THE INVENTION

The present invention relates to a device and method for facilitating collection of breast milk using a breast pump. More particularly, the invention relates to a device and method for promoting the let-down reflex in a nursing mother to facilitate collection of milk using a breast pump.

### BACKGROUND OF THE INVENTION

Let-down is the hormonal reflex in nursing mothers that causes milk to flow toward the milk ducts and nipples. This reflex is usually triggered by the sight of the woman's baby, or by its cries, and sometimes can be triggered by hearing or seeing any baby. In many cases, a woman can experience multiple let-down events during a single breastfeeding session. The experience of let-down is different for each woman, and the let-down reflex can be delayed or inhibited by stress, pain, embarrassment or other unpleasant feelings. In these cases, the milk flow may be interrupted temporarily, anywhere from a few minutes to several hours. When women experience difficulties in initiating milk flow when breastfeeding their babies, typical advice includes nursing the baby in a quiet location, making sure she is comfortable and relaxed, keeping hydrated, listening to music, along with many other suggestions to reduce stress. In most cases, the mere presence of the baby should eventually trigger the reflex. On the other hand, women frequently need to collect milk using a breast pump, for example, in cases where breastfeeding is not possible due to temporary separation of the baby and mother, or if regular pumping is needed to maintain milk supply as well as to collect breast milk. These women frequently experience problems with triggering the let-down reflex because their baby, the natural (psychological and emotional) trigger, is absent.

One approach that has been used to address this problem is to program an electronic breast pump with a pumping sequence that has variable suction pressures and cycles to simulate the initial sucking motion by an infant, which is usually fast and light, to stimulate the nipple and encourage let-down. Such a system is described in U.S. Pat. No. 6,547,756, U.S. Pat. No. 6,676,631, and U.S. Pat. No. 6,699,213, all assigned to Medela Holding AG, manufacturer of breast pumps sold under the trademarks LACTINA® and SYMPHONY® among others. The SYMPHONY® pump includes the programmable sequences to vary the pressure and cycling, including the sequence used to stimulate let-down. This device relies on physical sensations on the nipple induced by the vacuum pump to simulate the presence of the infant. Such an approach does not take into account the fact that each baby may have different levels of suction and different sucking speeds such that a one-size-fits-all sequence may not be sufficient to effectively trigger let-down in all women.

The most effective trigger for let-down in a woman who is nursing is her own baby, however, the prior art methods fail to take advantage of this natural trigger of the baby's audible crying to enhance milk production in a mother who wishes to provide breast milk for her baby. The present invention is directed to such a device and method.

### SUMMARY OF THE INVENTION

It is an advantage of the present invention to provide a breast pump assembly that enhances let down using the natural psychological and emotional triggers provided by a woman's infant.

In one aspect of the invention, an electronic breast pump assembly includes a housing that encloses a vacuum pump, a power supply, one or two connectors extending through the housing for attaching one or two breast shields to the vacuum pump, and an electronic recording device comprising a microphone, a memory for storing the sound received via the microphone, and a playback activator. Optionally, a pump controller with a memory can be included for entering and saving pump control programs for varying the level and rhythm of the vacuum applied through the breast shield(s).

In another aspect of the invention, the inventive breast pump assembly includes a vacuum pump in fluid communication with a breast shield for receiving a woman's breast and applying suction to draw milk from the breast and into a container and a sound module disposed in close proximity to the vacuum pump, the sound module comprising a speaker, a memory for storing an audio stream, a power supply and a playback actuator for playing the audio stream on demand, wherein the audio stream comprises one or more recordings of sounds made by a baby. In one embodiment, the sound module includes a microphone in electrical communication with the sound module and means for activating the microphone to record sounds made by the woman's baby for storage in the memory.

In yet another aspect of the invention, the breast pump assembly includes an electric vacuum pump in fluid communication with a breast shield, where the vacuum pump has variable suction and pumping cycles, a selector for selecting a suction level and suction frequency, and a sound module disposed in close proximity to the vacuum pump, the sound module comprising a microphone, speaker, a memory for storing an audio stream, a power supply, a recording actuator for activating the microphone to record the audio stream and a playback actuator for playing the audio stream on demand, wherein the audio stream comprises one or more recordings of sounds made by a baby. A controller may be provided in electrical communication with the vacuum pump for executing pre-programmed sequences of variable suction level and suction frequency. The controller may be additional operable to control one or both of recording and playback of the audio stream.

In an exemplary embodiment, the breast pump assembly includes a variable vacuum pump that is controlled by a programmable controller with a memory that stores pre-set or customizable sequences for producing varied suction levels and frequencies. The memory of the controller may also be used to store one or more audio recordings that are collected with a microphone included in the pump assembly. The user activates the microphone to record sounds made by the infant or toddler that are known or believed to induce let-down when heard by the mother. Alternatively, a separate sound chip or sound module may be used. Such sound chips, which are well known in the art and widely available from commercial sources, usually include a microphone and speaker so that a separate microphone and speaker will not be required. Sounds to be recorded can include crying, cooing, laughing, etc., i.e., any sound made by the child that the mother has determined to be a trigger for her own let-down reflex. Other sounds that provide the mother relaxation can be stored as well. In addition, or alternatively, a pre-recorded sound of an infant crying can also be stored. When the mother wishes to collect milk, she will perform the standard steps for setting up the breast pump assembly and breast shields to prepare for collection. Next, she presses a button, switch or other activator, to play back the recorded sound of her baby through a speaker located on or in the assembly's housing. The playback may be for a pre-set period of time, e.g., up to 30 seconds, as needed to initiate milk flow, or it can be played continuously while the mother collects her milk. The sound button can also be activated before the mother sets up the pump so as to have the milk ready to extract once the pump is set up an ready for use. In one embodiment, a headphone jack, which would bypass the speaker when headphones are connected, can be included to allow the mother to listen to her child's sounds without disturbing others who may be nearby. This would be useful, for example, if the mother is pumping in a corporate or work environment. In another embodiment, multiple sounds may be recorded to first enhance the let-down reflex, e.g., the baby's cry when he or she is hungry, and then to relax the mother while expressing the milk, e.g., the baby's cooing sounds while nursing.

Additional audio capabilities may be provided by including a dock for an iPOD®, MP3, iPHONE® or similar electronic player to allow music or other sound recordings to be played through the speaker on the assembly. Such devices also provide for the inclusion of visual stimuli to enhance let-down and/or relaxation because the player may be used to display a photograph or video of the baby that the mother can view while collecting her milk. The player may also contain visual and auditory instructional material on how to use the sound button or other controls of the pump.

### BRIEF DESCRIPTION OF THE DRAWINGS

FIG. 1 is a perspective view of an exemplary embodiment of the invention.

FIG. 2 is a diagrammatic front view of the embodiment of FIG. 1.

FIG. 3 is a block diagram showing the components of a breast pump assembly according to one embodiment of the present invention.

FIG. 4 is a perspective view of an alternative embodiment of the invention for manual pumping.

### DETAILED DESCRIPTION OF THE EXEMPLARY EMBODIMENTS

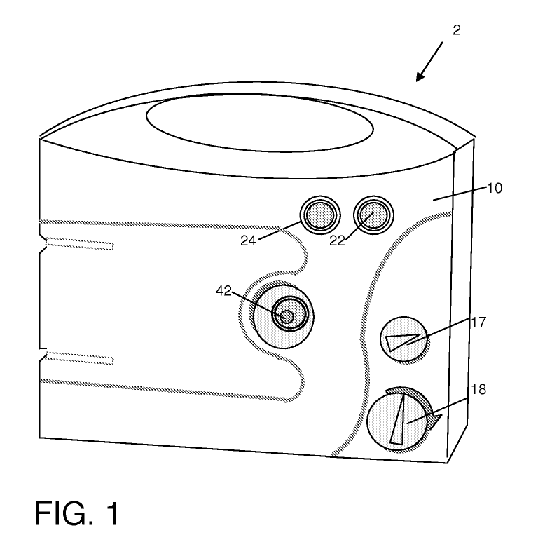

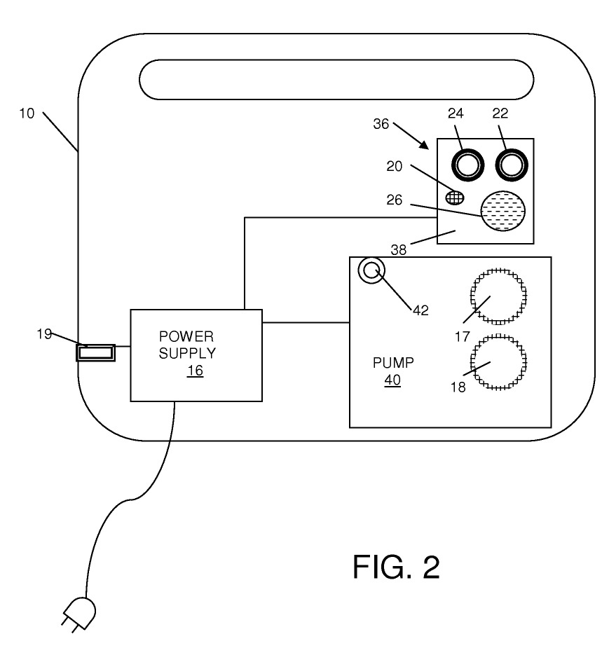

FIGS. 1 and 2 illustrate a first embodiment of the invention incorporated into an electronic breast pump assembly 2. The breast pump assembly 2 includes a housing 10, a vacuum level adjustment knob 17 and a vacuum cycling frequency adjustment knob 18, a record button 24, a playback button 22 and one or two pump connectors 42 for attaching vacuum tubing to the pump 40. (Only one connector 42 is shown.) It should be noted that the knobs, buttons and switches that are described herein may be replaced by other appropriate actuators without deviating from the scope of the invention.

As can be seen more clearly in FIG. 2, knobs 17 and 18 directly control the pump 40, which may be a diaphragm pump, a piston pump, or any other type of pump that is known in the art to be suitable for use in a breast pump. The pump 40 receives power to drive its operation from power supply 16, which may be connected to an external voltage source at connecter 19, to a battery pack that supplies a D.C. voltage. e.g., 12 VDC, or to a wall outlet for operation on 100-240 VAC, 50-60 Hz, in which case the power supply will include an AC/DC converter to produce an appropriate DC voltage to drive the pump.

A sound module 36 includes, a speaker 26, a memory and appropriate circuitry on a printed circuit board 38. A microphone 20 may also be included in the module, or may be a separate component connected via appropriate means. Sound modules, such as those that are included in greeting cards and toys, are commercially available. Buttons 22 and 24, or other actuators, are electrically connected to the sound module 36 to activate the record and playback functions, respectively, of the module. Many sound modules include their own lithium ion button battery, which may be used, or the module will preferably be connected to the power supply 16 with appropriate circuitry to provide the reduced voltage required by the module so that battery replacement will not be required after many uses.

Prior to utilizing the let-down enhancing feature of the breast pump, the mother may wish to observe which sounds made by her baby tend to trigger the let-down reflex since the stimulus will be different for different women. After determining the most effective sound, with her baby in close proximity to the breast pump, the mother will press button 22 to initiate recording of the sounds made by her baby. Such sounds may include crying, cooing, the sounds of suckling, laughter, or any other sound that the baby makes that most quickly triggers let-down. Most commonly-available sound modules are capable of recording a 30 second audio stream, however, it may be desirable to provide the capability for a longer recording or a loop in case the woman wishes to have continuous sound to assist in her relaxation while collecting milk after let-down has occurred.

In an alternative embodiment, the sound module 36 may include a pre-recorded baby's cries which may be used by women whose let-down reflex occurs in response to hearing any baby's cry. In this case, a microphone need not be included.

To begin collection of milk, the mother will set up the breast feeding kit, connecting the tubing to connector(s) 42 for use of one or two breast shields. After getting comfortable, just prior to, or at the same time she activates the pump 40, she presses the playback button 24 to play the sound of her baby that she recorded previously. This should trigger the let-down reflex to allow her to immediately begin collecting milk. If let-down does not occur, she may wish to repeat the playback operation by again pressing button 24. In one embodiment, the playback button 24 may include a two-position switch that, when pressed twice, causes continuous playback of the recorded sound, and which may then be pressed again to discontinue playback once let-down has occurred, or when milk collection is complete. The mother can also trigger the let-down button before she sets up the kit to initiate the let-down process and ensure that the breast is ready to be pumped when the set up is finished. This has the advantage of decreasing the overall pumping time.

In one embodiment, the breast pump assembly is configured for portability, including being small (40 cm or less in its largest dimension, more preferably, 20 cm or less) and relatively lightweight (7 kg or less, more preferably, 2 kg or less). The housing 10 of the breast pump assembly 2 includes a carrying handle, which may be integrally formed in the housing mold, as shown, or may be attached to the housing via appropriate fasteners. However, portability is not essential, and the pump could be of larger design such that it would have longer durability.

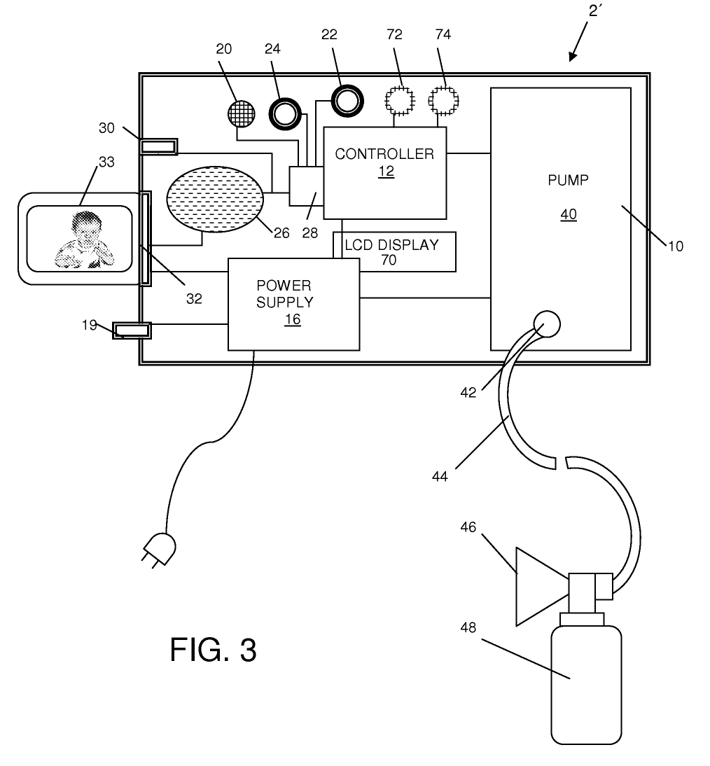

FIG. 3 illustrates an alternative embodiment of the breast pump assembly 2′ which incorporates all elements of the embodiment of FIGS. 1 and 2 and adds a controller 12 with a memory that is capable of storing and executing programmed sequences for varying vacuum pressure and cycling. The sequences can be factory pre-programmed and, for example, selected by program number, or custom sequences can be entered and saved based on settings that the mother finds to be most comfortable and effective for milk collection. Controller 12 is connected to sound module 28 and may be used to control operation (recording and playback) of the audio signals, including repeating recordings, playing the recording for pre-set time period, or playing different recordings at different stages during a breast pumping session. The pump's settings are selected by or through controller 12, with knobs 72 and 74 being used to select pumping pressure, pump cycling (frequency of application of suction), or for selecting the sequence number of a pre-programmed sequence. An optional LCD or similar display 70 is shown connected to controller 12 for display of one or more of the operating parameters, selected sequence number, a timer, and any number of messages that may be useful to the mother during set up and milk collection using the breast pump.

An optional feature of the breast pump assembly 2′ is an MP3 docking station 32, which may be used to connect an iPOD®, iPHONE®, MP3 player 33, or other similar electronic device or mobile phone that is capable of recording and playback of music and other audio signals, video and photographs. For purposes of this description, all such devices, whether a recorder/player or a mobile phone, will be referred to as an “MP3 player”. The mother may choose to further enhance let-down by using the MP3 player to display a photograph, a slide show or a video of her baby in combination with the audio stimulation. The MP3 docking station 32 may be connected to the assembly's speaker 26 or headphones (not shown) if headphones are plugged into headphone jack 30, so that the music or other recording can be played back while the woman collects her milk without tying up the woman's hands trying to operate the player separately. In a preferred embodiment, the controller 12 may include the capability to interact with the MP3 player, allowing volume or other controls to be selected via the knobs, buttons and/or switches on the pump housing. The power supply 16 of the breast pump assembly provides power to the MP3 docking station 32 so that the battery of the MP3 player does not run down during use.

The MP3 docking station may contain independent programs applications, or applets, that can control certain aspects of the pump such as the letdown button. The applet that is running can control the letdown function by triggering the recorded baby's cry or other sound. The applet may control other aspects of the pump to facilitate letdown such as the cycling speed (suction frequency) or strength (suction level) of the pump.

Controller 12 is connected to pump 40 to control pumping pressures and cycles. Vacuum tubing 44 is connected at its proximal end to connector 42 and at its distal end to breast shield assembly 46 which directs expressed milk into collection container 48. For double pumping, two connectors 42 may be used, or a t-connector may be inserted into the line 44 to allow two separate lines to be attached to two separate breast shield assemblies.

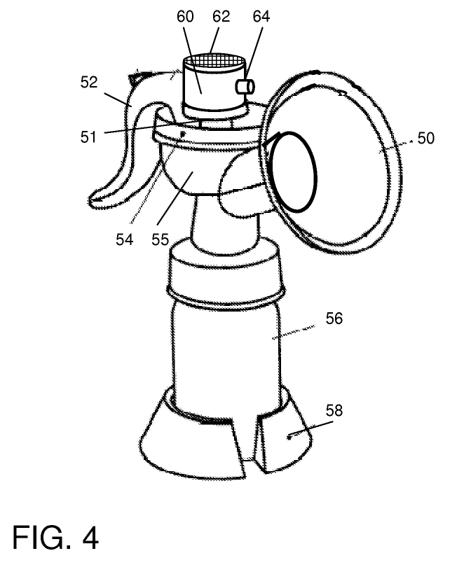

FIG. 4 illustrates an alternative embodiment of the invention for use with a hand breast pump. The breast shield assembly includes breast shield 50 which is in fluid communication with cup 55. Diaphragm 54 covers the top of cup 55 with piston 51 connected to its center. When lever 52 is depressed, piston 51 pulls up on diaphragm 54 to create suction within cup 55 which is communicated to shield 50. The suction draws milk from the woman's breast when the shield is held against the breast to form an airtight seal. The expressed milk passes down the neck of the breast shield assembly to be collected in container 56. Container 56 is releasably supported by base 58.

Disposed on top of piston 51 is a sound module 60 with an internal microphone, circuitry and battery. The module 60 should be enclosed within a protective casing to prevent damage to the module's components. As in the previously-described embodiments, the mother records the desired sound made by her baby, which is stored within the memory of the sound module 60. Button 64 may be used to activate the recording function as well as being used for playback, or separate buttons may be provided for each operation. When the mother is ready to begin pumping, she presses button 64 to play back the recording of her child, thus enhancing let-down. As will be apparent to those in the art, the sound module 60 can be attached to any area of the breast pump where it can be positioned reasonably out of the way during normal use, including the neck or lever, or to the base 58. Furthermore, the sound module may be used with different types of manual pumps and is not limited to the pump configuration illustrated. In one embodiment, the sound module 60 is releasably attached to the pump, e.g., by a hook-and-pile fastener, a snap or other conventional attachment means, to allow the module to be removed prior to washing of the pump. Alternatively, the sound module can be fully sealed within a resilient elastomer or similar material that is used to form, for example, the base 58 or the bottom of the collection container. Compression of the elastomer by pressing the collection container 56 down against the center of the base 58, would activate the switch for recording and playback. This would allow the base to be washed without damaging the sound module.

The present invention provides a device and method for using natural psychological and emotional triggers to enhance the let-down reflex in nursing mothers who use breast pumps to collect their milk. The device can be readily attached to an existing breast pump (electric or manual) using a conventional sound module for recording and on-demand play of sounds made by the baby that initiate let-down in the mother. Preferably, however, the device is incorporated internally into the breast pump assembly, with switches and other controllers to allow customization of the audio stimulation to optimize let-down and milk expression. Additional features can include visual stimulation that can be used in combination with the audio playback.

Various modifications to the above-described embodiments will be readily apparent to those skilled in the art, and the generic principles described herein can be applied to other embodiments without departing from the spirit or scope of the invention. Thus, it is to be understood that the description and drawings presented herein represent presently preferred embodiments of the invention and are therefore representative of the subject matter which is broadly contemplated by the present invention. Accordingly, the scope of the present invention is limited by nothing other than the appended claims.

1. A breast pump assembly comprising:
a vacuum pump in fluid communication with a breast shield for receiving a woman's breast and applying suction to draw milk from the breast and into a container; and
a sound module disposed in close proximity to the vacuum pump for storing and playing back an audio stream comprising one or more recordings of sounds made by a baby.

2. The breast pump assembly of claim 1, wherein the sound module includes a microphone and means for activating the microphone to record sounds made by the woman's baby for storage in the sound module.

3. The breast pump assembly of claim 1, wherein sound module includes a playback actuator having a two position switch to select single playback or continuous playback of the audio stream.

4. The breast pump assembly of claim 1, wherein the vacuum pump is an electric pump disposed within a housing and the sound module is disposed on or in the housing.

5. The breast pump assembly of claim 4, wherein the electric pump has variable suction and pumping cycles, and further comprising user controls for selecting a suction level and suction frequency.

6. The breast pump assembly of claim 5, wherein the electric pump has variable suction and pumping cycles, and further comprising a controller for executing pre-programmed sequences of variable suction level and suction frequency.

7. The breast pump assembly of claim 6, wherein the controller further operates to control one or both of recording and playback of the audio stream.

8. The breast pump assembly of claim 4, further comprising a headphone jack for bypassing a speaker in the sound module when headphones are plugged into the jack.

9. The breast pump assembly of claim 4, further comprising a MP3 docking station for connection of a MP3 player for playback of one or both of a pre-recorded audio stream and a visual image of the woman's baby.

10. The breast pump assembly of claim 9, wherein the visual image is a video recording.

11. The breast pump assembly of claim 9, wherein the MP3 player contains a software applet for triggering one or more of the sound module and variable suction levels and suction frequency.

12. The breast pump assembly of claim 1, wherein the vacuum pump is a manual pump and the sound module is disposed on an outer surface of the pump.

13. A breast pump assembly comprising:
a vacuum pump in fluid communication with a breast shield for receiving a woman's breast and applying suction to draw milk from the breast and into a container; and
a sound module disposed in close proximity to the vacuum pump, the sound module comprising a speaker, a memory for storing an audio stream, a power supply and a playback actuator for playing the audio stream on demand, wherein the audio stream comprises one or more recordings of sounds made by a baby.

14. The breast pump assembly of claim 13, further comprising a microphone in electrical communication with the sound module and means for activating the microphone to record sounds made by the woman's baby for storage in the memory.

15. The breast pump assembly of claim 13, wherein the playback actuator comprises a two position switch to select single playback or continuous playback of the audio stream.

16. The breast pump assembly of claim 13, wherein the vacuum pump is an electric pump disposed within a housing and the sound module is disposed on or in the housing.

17. The breast pump assembly of claim 16, wherein the electric pump has variable suction and pumping cycles, and further comprising user controls for selecting a suction level and suction frequency.

18. The breast pump assembly of claim 17, wherein the electric pump has variable suction and pumping cycles, and further comprising a controller for executing pre-programmed sequences of variable suction level and suction frequency.

19. The breast pump assembly of claim 18, wherein the controller further operates to control one or both of recording and playback of the audio stream.

20. The breast pump assembly of claim 15, further comprising a headphone jack for bypassing the speaker when headphones are plugged into the jack.

21. The breast pump assembly of claim 15, further comprising a MP3 docking station for connection of a MP3 player for playback of one or both of a pre-recorded audio stream and a visual image of the woman's baby.

22. The breast pump assembly of claim 21, wherein the visual image is a video recording.

23. The breast pump assembly of claim 21, wherein the MP3 player contains a software applet for triggering one or more of the sound module and variable suction levels and suction frequency.

24. The breast pump assembly of claim 12, wherein the vacuum pump is a manual pump and the sound module is disposed on an outer surface of the pump.

25. A breast pump assembly comprising:
an electric vacuum pump in fluid communication with a breast shield for receiving a woman's breast and applying suction to draw milk from the breast and into a container, wherein the vacuum pump has variable suction and pumping cycles;
a selector for selecting a suction level and suction frequency; and
a sound module disposed in close proximity to the vacuum pump, the sound module comprising a microphone, speaker, a memory for storing an audio stream, a power supply, a recording actuator for activating the microphone to record the audio stream and a playback actuator for playing the audio stream on demand, wherein the audio stream comprises one or more recordings of selected sounds made by the woman's baby, wherein the sounds are selected according to their effectiveness for triggering let-down.

26. The breast pump assembly of claim 25, further comprising a controller in electrical communication with the vacuum pump for executing pre-programmed sequences of variable suction level and suction frequency.

27. The breast pump assembly of claim 26, wherein the controller further operates to control one or both of recording and playback of the audio stream.

28. The breast pump assembly of claim 25, further comprising a headphone jack for bypassing the speaker when headphones are plugged into the jack.

29. The breast pump assembly of claim 25, further comprising a MP3 docking station for connection of a MP3 player for playback of one or both of a pre-recorded audio stream and a visual image of the woman's baby.

30. The breast pump assembly of claim 29, wherein the visual image is a video recording.

31. The breast pump assembly of claim 29, wherein the MP3 player contains a software applet for triggering one or more of the sound module and variable suction levels and suction frequency.

32. A breast pump assembly comprising:
a housing;
an electric vacuum pump disposed within the housing, the vacuum pump having variable suction and pumping cycles;
at least one connector for connecting vacuum tubing attached to a breast shield for receiving a woman's breast and applying suction to draw milk from the breast and into a container;
at least selector for selecting a suction level and suction frequency;
a sound module disposed within the housing, the sound module comprising a microphone, speaker, a memory for storing an audio stream, a recording actuator for activating the microphone to record the audio stream and a playback actuator for playing the audio stream on demand, wherein the audio stream comprises one or more recordings of selected sounds made by the woman's baby, wherein the sounds are selected according to their effectiveness for triggering let-down; and
a power supply for providing power to the electric pump, the controller and the sound module.

33. The breast pump assembly of claim 32, further comprising a controller disposed within the housing in electrical communication with the vacuum pump for executing pre-programmed sequences of variable suction level and suction frequency.

34. The breast pump assembly of claim 33 wherein the controller further operates to control one or both of recording and playback of the audio stream.

35. The breast pump assembly of claim 32, further comprising a headphone jack for bypassing the speaker when headphones are plugged into the jack.

36. The breast pump assembly of claim 32, further comprising a MP3 docking station for connection of a MP3 player for playback of one or both of a pre-recorded audio stream and a visual image of the woman's baby.

37. The breast pump assembly of claim 36, wherein the visual image is a video recording.

38. The breast pump assembly of claim 36, wherein the MP3 player contains a software applet for triggering one or more of the sound module and variable suction levels and suction frequency.
